(19) United States
(12) Patent Application Publication
(10) Pub. No.: US 2024/0166696 A1
(43) Pub. Date: May 23, 2024

(54) Scaffolds For Inducing Antibody Responses Against Antigenic Sites

(71) Applicant: Emory University, Atlanta, GA (US)

(72) Inventors: Chinglai Yang, Decatur, GA (US); Ling Ye, Decatur, GA (US); Samuel Stampfer, Atlanta, GA (US); Rui Jin, Decatur, GA (US)

(21) Appl. No.: 18/279,726

(22) PCT Filed: Mar. 8, 2022
(86) PCT No.: PCT/US2022/019230

§ 371 Date: Aug. 31, 2023

Related U.S. Application Data
(60) Provisional application No. 63/157,892, filed on Mar. 8, 2021.

(51) Int. Cl. C07K 14/08; A61K 38/00; A61P 31/14; A61P 37/04; C12N 15/63

(52) U.S. Cl. CPC C07K 14/08; A61K 38/00; A61P 31/14; A61P 37/04; C12N 15/63; C12N 2760/14122; C12N 2770/20022

(57) ABSTRACT

This disclosure relates to scaffolds for inducing antibody responses against antigenic sites. In certain embodiments, this disclosure relates to compositions and methods using a filovirus sGP as a scaffold for inducing antibody responses against antigenic sites in foreign pathogens. In certain embodiments, this disclosure relates to compositions and methods using a filovirus sGP as a scaffold for inducing antibody responses against a virus to produce a viral vaccine.

### CROSS-REFERENCE TO RELATED APPLICATIONS

This application claims the benefit of U.S. Provisional Application No. 63/157,892 filed Mar. 8, 2021. The entirety of this application is hereby incorporated by reference for all purposes.

### STATEMENT REGARDING FEDERALLY SPONSORED RESEARCH OR DEVELOPMENT

This invention was made with government support under AI111851 awarded by the National Institutes of Health. The government has certain rights in this invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 21013PCT_ST25.txt. The text file is 43 KB, was created on Mar. 8, 2022, and is being submitted electronically via EFS-Web.

### BACKGROUND

Some common colds are due to certain coronavirus (CoV) strains associated with mild symptoms. More dangerous human strains such as severe acute respiratory syndrome associated coronavirus (SARS-CoV-1) and SARS-CoV-2 (also referred to as COVID-19) are believed to result from coronavirus strains jumping to humans by secondary zoonotic transfers, e.g., from bats to cats and cats to humans. In humans, SARS-CoV-2 can be transferred from individuals who have mild symptoms or are asymptomatic and has caused numerous deaths worldwide. Thus, there is a need to identify treatments and preventative measures.

Rottier et al. report coronavirus-like particles comprising functionally deleted genomes. US 2004/0071709.

Martinez et al. report an impact of ebola mucin-like domain on antiglycoprotein antibody responses induced by ebola virus-like particles. The Journal of Infectious Diseases, 2011, 204: S825-S832.

Baric et al. report methods and compositions for coronavirus diagnostics and therapeutics. WO 2015/057666.

Yao et al. report an ebola virus and marburg virus glycoprotein mucin-like domain replacement expression system used as a vaccine approach. WO 2019/113688.

References cited herein are not an admission of prior art.

### SUMMARY

This disclosure relates to scaffolds for inducing antibody responses against antigenic sites. In certain embodiments, this disclosure relates to compositions and methods using a filovirus sGP as a scaffold for inducing antibody responses against antigenic sites in foreign pathogens. In certain embodiments, this disclosure relates to compositions and methods using a filovirus sGP as a scaffold for inducing antibody responses against coronavirus to produce a coronavirus vaccine, e.g., SARS-Cov-2 vaccine.

In certain embodiments, this disclosure relates to fusion proteins, or vectors encoding the same, comprising a heterologous sequence inserted in the middle of a filovirus secreted glycoprotein (sGP). In certain embodiments, the filovirus is selected form Ebola virus (EBOV), Sudan virus (SUDV); Bundibugyo virus (BDBV); Tai Forest virus (TAFV); Reston virus (RESTV); Marburg virus (MARV); and Lloviu virus (LLOV).

In certain embodiments, the fusion protein, nucleic acids, or vectors encoding the same, have a heterologous sequence inserted between amino acids corresponding to amino acids 188 and 213 of Ebola sGP.

In certain embodiments, the fusion protein, nucleic acids, or vectors encoding the same, have a heterologous sequence inserted after the amino acid corresponding to amino acid 300 in the Ebola virus sGP.

In certain embodiments, the fusion protein, nucleic acids, or vectors encoding the same, have a heterologous sequence which is a microbial sequence, viral sequence, bacterial sequence, or parasite sequence.

In certain embodiments, the fusion protein, nucleic acids, or vectors encoding the same, have a viral sequence which is a viral spike protein sequence, a viral heptad repeat (HR) region sequence, a viral HR1 (heptad repeat 1) region sequence, a viral HR2 (heptad repeat 2) region sequence, or a viral membrane-proximal extracellular region (MPER) sequence, or a viral surface glycoprotein sequence.

In certain embodiments, the fusion protein, nucleic acids, or vectors encoding the same, have a viral sequence which is a coronavirus sequence, an influenza virus sequence, an influenza virus hemagglutinin (HA) or neuraminidase (NA) sequence, a Lassa Fever virus sequence, a Lassa Fever virus F protein sequence, a human immunodeficiency virus sequence, a human immunodeficiency virus glycoprotein gp160 sequence, a respiratory syncytial virus sequence, a respiratory syncytial virus surface glycoproteins F or HN sequence, a Nipah virus sequence, a Nipah virus surface glycoprotein G or F sequence, a Hendra virus sequence, a Hendra virus surface glycoproteins G and F, or fragments thereof. In certain embodiments, the fragment is more than 4, 5, 6, 7, 8, 9, or 10 amino acids in length.

In certain embodiments, the fusion protein has a linker comprising glycine and/or serine between a filovirus sequence and the heterologous sequence or coronavirus sequence.

In certain embodiments, the heterologous sequence is a coronavirus sequence. In certain embodiments, this disclosure relates to fusion proteins, nucleic acids, or vectors encoding the same, wherein the fusion protein comprises or consists of the amino acid sequences disclosed herein such as SEQ ID NO: 1, 5, 9, 12, 17, 19, 20 23, 24, 25, 26, 27, 28, or 29, or variants thereof.

In certain embodiments, the fusion protein, nucleic acids, or vectors encoding the same, comprises or consists of the amino acid sequence of MGVTGILQLPRDRFKRTSFFLWVIILFQRTFSIPLGVIHNSTLQVSDVDKLVCRDKLSSTN QLRSVGLNLEGNGVATDVPSATKRWGFRSGVPPKVVNYEAGEWAENCYNLEIKKPDG SECLPAAPDGIRGFPRCRYVHKVSGTGPCAGDFAFHKEGAFFLYDRLASTVIYRGTTFAE GVVAFLILPQAKKDFFSSHPLREPVNATEDPSSGYYSTTIRYQATGFGTNETEYLFEVDN LTYVQLESRFTPQFLLQLNETIYTSGKRSNTTGKLIWKVNPEIDTTIGERSFIEDLLFNKVT LADAGKFAVKSCLSQLYQTEPKTSVV (SEQ ID NO: 1) (Group 1: FP-end) or variant thereof.

In certain embodiments, this disclosure relates to fusion proteins, nucleic acids, or vectors encoding the same, wherein the fusion protein comprises or consists of the amino acid sequence of
MGVTGILQLPRDRFKRTSFFLWVIILFQRTFSIPLGVIHNSTLQVSDVDKLVCRDK LSSTNQLRSVGLNLEGNGVATDVPSATKRWGFRSGVPPKVVNYEAGEWAENCYNLEIK KPDGSECLPAAPDGIRGFPRCRYVHKVSGTGPCAGDFAFHKEGAFFLYDRLASTVIYRG TTFAEGVVAFLILPSKRSFIEDLLFNKVTLADAGPSSGYYSTTIRYQATGFGTNETEYLFE VDNLTYVQLESRFTPQFLLQLNETIYTSGKRSNTTGKLIWKVNPEIDTTIGEWAFWETKK TSLEKFAVKSCLSQLYQTEPKTSVV (SEQ ID NO: 5) (Group 2: FP-mid) or variant thereof.

In certain embodiments, this disclosure relates to fusion proteins, nucleic acids, or vectors encoding the same, wherein the fusion protein comprises or consists of the amino acid sequence of
MGVTGILQLPRDRFKRTSFFLWVIILFQRTFSIPLGVIHNSTLQVSDVDKLVCRDK LSSTNQLRSVGLNLEGNGVATDVPSATKRWGFRSGVPPKVVNYEAGEWAENCYNLEIK KPDGSECLPAAPDGIRGFPRCRYVHKVSGTGPCAGDFAFHKEGAFFLYDRLASTVIYRG TTFAEGVVAFLILPENQKLIANQFNSAIATEDPSSGYYSTTIRYQATGFGTNETEYLFEVD NLTYVQLESRFTPQFLLQLNETIYTSGKRSNTTGKLIWKVNPEIDTTIGERSFIEDLLFNKV TLADAGKFAVKSCLSQLYQTEPKTSVV (SEQ ID NO: 9) (Group 3: HR1/FP) or variant thereof.

In certain embodiments, this disclosure relates to fusion proteins, nucleic acids, or vectors encoding the same, wherein the fusion protein comprises or consists of the amino acid sequence of
MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL ASKRSFIEDLLFNKVTLADAGTSSYYATSYLEYEIENFGAQHSTTLFKIDNNTFVRLDRPH TPQFLFQLNDTIHLHQQLSNTTGRLIWTLDANINADIGERSFIEDLLFNKVTLADAGKFAV KSCLSQLYQTEPKTSVV (SEQ ID NO: 12)(SUDV FP/FP) or variants thereof.

In certain embodiments, this disclosure relates to fusion proteins, nucleic acids, or vectors encoding the same, wherein the fusion protein comprises or consists of the amino acid sequence of
MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL AKPKETFLQSPPIREAVNYTENTSSYYATSYLEYEIENFGAQHSTTLFKIDNNTFVRLDRP HTPQFLFQLNDTIHLHQQLSNTTGRLIWTLDANINADIGERSFIEDLLENKVTLADAGNY VEKSCLSKLYRSTRQKTMMR (SEQ ID NO: 17)(Grp 1: SUDV FP-end) or variants thereof.

In certain embodiments, this disclosure relates to fusion proteins, nucleic acids, or vectors encoding the same, wherein the fusion protein comprises or consists of the amino acid sequence of
MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL ASKRSFIEDLLFNKVTLADAGTSSYYATSYLEYEIENFGAQHSTTLFKIDNNTFVRLDRPH TPQFLFQLNDTIHLHQQLSNTTGRLIWTLDANINADIGERSFIEDLLENKVTLADAGNYV EKSCLSKLYRSTRQKTMMR (SEQ ID NO: 19)(Grp 2: SUDV FP/FP) or variants thereof.

In certain embodiments, this disclosure relates to fusion proteins nucleic acids, or vectors encoding the same, wherein the fusion protein comprises or consists of the amino acid sequence of
MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL ASKRSFIEDLLFNKVTLADAGTSSYYATSYLEYEIENFGAQHSTTLFKIDNNTFVRLDRPH TPQFLFQLNDTIHLHQQLSNTTGRLIWTLDANINADIGEWAFWENKKISPNNYVEKSCLS KLYRSTRQKTMMR (SEQ ID NO: 20)(Grp 3:SUDV FP-mid) or variants thereof.

In certain embodiments, this disclosure relates to fusion proteins, nucleic acids, or vectors encoding the same, wherein the coronavirus sequence comprises an amino acid sequence selected from RSFIEDLLFNKVTLADAG (SEQ ID NO: 4), ENQKLIANQFNSAI (SEQ ID NO: 11), and LNESLIDLQELGKYE (SEQ ID NO: 22), or variants with one, two, or three amino acid substitutions or greater than 60%, 70%, 80%, or 90% identity.

In certain embodiments, this disclosure relates to nucleic acid encoding a fusion protein disclosed herein in operable combination with a promoter, wherein the fusion protein comprise a heterologous sequence inserted in the middle of filovirus soluble glycoprotein (sGP). In certain embodiments, this disclosure relates to a vector comprising a nucleic acid encoding a fusion protein disclosed herein. In certain embodiments, the nucleic acid or vector is DNA or RNA.

In certain embodiments, the variant of a fusion protein disclosed herein has greater than 70% 80%, 90%, 95%, 96%, 97%, 98%, 99%, identity to a sequence disclosed herein.

In certain embodiments, this disclosure relates to virus particles or virus like particles comprising a fusion protein comprising a heterologous or coronavirus sequence as disclosed herein, e.g., inserted between amino acids corresponding to amino acids 188 and 213 of Ebola sGP.

In certain embodiments, this disclosure relates to expression system comprising a vector or nucleic acid encoding a fusion protein in operable combination with a promoter, wherein the fusion protein comprising a heterologous sequence or coronavirus sequence as disclosed herein, e.g., inserted in the middle of filovirus soluble glycoprotein (sGP) corresponding to amino acids 188 and 213 of sGP of Ebola.

In certain embodiments, this disclosure relates to pharmaceutical compositions and vaccine compositions comprising a fusion protein, nucleic acid, or vector encoding the same, as disclosed herein and a pharmaceutically acceptable carrier optionally further comprising an adjuvant. In certain embodiments, the adjuvant is aluminum hydroxide, aluminum phosphate, aluminum potassium sulfate, monophosphoryl lipid A, oil in water emulsion composed of squalene, QS-21, a natural compound extracted from the Chilean soapbark tree, unmethylated cytosine-phosphate-guanosine oligodeoxynucleotides, or combinations thereof.

In certain embodiments, this disclosure relates to methods of treating or preventing a viral infection or coronavirus infection or reducing the symptoms of a viral or coronavirus infection comprising administering to a subject in need thereof an effective amount of a fusion protein, nucleic acid or vector encoding the same, as disclosed herein optionally in combination with an adjuvant.

In certain embodiments, this disclosure relates to methods of treating or preventing a microbial or viral infection or reducing the symptoms of a microbial infection, viral infection or coronavirus infection comprising administering to a subject in need thereof an effective amount of a nucleic acid or vector encoding a fusion protein in operable combination with a promoter, wherein the fusion protein is as disclosed herein and optionally the nucleic acid or vector is administered in combination with an adjuvant. In certain embodiments the nucleic acid is DNA or RNA.

In certain embodiments, this disclosure relates to methods of treating or preventing a microbial infection, viral infection, or coronavirus infection or reducing the symptoms of a viral infection or coronavirus infection comprising administering to a subject in need thereof an effective amount of a virus particle or virus like particle comprising a fusion protein as described herein.

In certain embodiments, this disclosure relates to the production of medicaments for use in vaccinations or treatments disclosed herein having compositions disclosed herein.

### BRIEF DESCRIPTION OF THE SEVERAL VIEWS OF THE DRAWINGS

FIG. 1 illustrates the EBOV sGP is synthesized as a single polypeptide which forms a dimer structure after synthesis. EBOV sGP is the major glycoprotein produced during virus infection which is efficiently secreted from cells after synthesis.

FIG. 2 illustrates exposure to sGP by immunizations could exert a dominant effect on the profiles of antibody responses induced by vaccination. In experiments for vaccine development against Ebola virus (EBOV) infection, the viral sGP (secreted glycoprotein) exhibits strong immunogenicity in vaccinated animals.

FIG. 3 illustrates the SARS-COV-2 S protein (spike protein) for identification of potential conserved and exposed epitopes for vaccine development. The structure of the SARS-COV-2 S protein (based on PDB ID 6VSB) was analyzed along with sequence homology analysis for various strains of lineage B β-coronavirus, including SARS-COV, SARS-COV2, as well as other SARS-related β-coronaviruses. Through sequence comparison, three segments in the S2 subunit: RSFIEDLLFNKVTLADAGF (SEQ ID NO: 21), ENQKLIANQFNSAI (SEQ ID NO: 11), and LNESLIDLQELGKYE (SEQ ID NO: 22); were found to show high homology among all these lineage B β-coronavirus (from 92% to 100% in homology) and also found to be exposed in the S protein trimer, making them possible targets for vaccine development. The chimeric protein antigens with conserved targets from the SARS-COV-2 S protein FP, HR1, and HR2/MPR were engrafted onto the Ebola virus sGP protein.

FIG. 4 shows data on inducing antibodies against the fusion peptide (FP) of the SARS-COV-2 S protein by sGP-FP chimeric protein DNA vaccine. Mice (groups of 5) were vaccinated with DNA vaccines expressing the sGP-FP chimeric protein or the wild type EBOV sGP. Immunization was carried by intramuscular injection of 100 ug DNA vaccines, and mice were vaccinated twice at a 4-week interval. At 2 weeks after the second immunization, blood samples were collected, and the levels of antibody responses against the fusion peptide of the SARS-COV-2 S protein were determined by ELISA using a synthetic peptide corresponding to the FP of the SARS-COV-2 S protein. The results shown in the graph are the O.D. values of serum samples from each mouse at 1:50 dilution.

FIG. 5 shows data on the reactivity of antibodies induced by sGP-FP to the whole SARS-COV2 Spike protein by ELISA. Mice (groups of 5) were vaccinated with DNA vaccines expressing the sGP-FP chimeric proteins (including three different designs designated as, FP-end, FP-mid, or HR1/FP respectively as illustrated in FIG. 11) or the wild-type EBOV sGP. Immunization was carried by intramuscular injection of 100 ug DNA vaccines, and mice were vaccinated twice at a 4-week interval. The mice were given the third immunization 4 weeks later with a DNA vaccine expressing SUDV FP/FP. At 2 weeks after the third immunization, blood samples were collected, and the levels of antibody responses against the Spike protein (S) of the SARS-COV-2 were determined by ELISA, using insect cell supernatants containing SARS-COV-2 S protein. The results shown in the graph are the O.D. values of serum samples from each mouse at 1:50 dilution and 1:200 dilution.

FIG. 6 shows ELISA data on 100 ng/well of pure spike protein for three coronaviruses: SARS-COV-2 (labeled “COVID), SARS-CoV (labeled “SARS), and MERS-CoV (labeled “MERS”)—sera of individual mice that were immunized with twice with the Ebola sGP FP-Mid, followed by a boosting immunization with the DNA vaccine SUDV sGP FP/FP, and then a boosting immunization by a purified protein vaccine SUDV sGP FP/FP. The serum samples were collected on day 20 after the fourth immunization (designated as V4D20).

FIG. 7 shows ELISA data on 50 nanograms/well of pure MERS or COVID spike protein, evaluating individual mouse sera from all immunization groups. The serum samples were collected on day 20 after the fourth immunization (designated as V4D20).

FIG. 8 shows protein sequences (SEQ ID NO: 23, 24, and 25) of constructs used in the first two immunizations, DNA vaccines. FP-mid is the construct above with fusion peptide in the middle of sGP. FP-end has the fusion peptide inserted near the end of sGP, with FP amino acids 815-832 replacing sGP residues 288-299. HR1/FP has residues from hairpin 1 (HR1) of COVID spike inserted into the middle of sGP and residues from FP of COVID spike inserted into the end (FP 815-832 replaces sGP 288-299; HR1 918-930 replaces sGP 188-204).

FIG. 9 shows a protein sequence (SEQ ID NO: 26) of a construct used in the third immunization, DNA route: SUDV FP/FP, N-terminal Melittin signal sequence was used to enhance insect cell expression; last 20 bases are from Ebola sGP instead of Sudan sGP.

FIG. 10 shows protein sequences (SEQ ID NO: 27, 28, and 29) of constructs used for the fourth immunization.

FIG. 11 illustrates the DNA constructs that had been used as DNA vaccines directly or to produce purified protein vaccines.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. Thus, reference to an “embodiment” refers to an example of the invention and is not necessarily limited by such an example.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a rage of amino acids expressly specified in the claim. In certain embodiments, the disclosure contemplates that the “N-terminus of a peptide may consist of an amino acid sequence,” which refers to the N-terminus of the peptide having the exact number of amino acids in the sequence and not more or having not more than a rage of amino acids specified in the claim however the C-terminus may be connected to additional amino acids, e.g., as part of a larger peptide. Similarly, the disclosure contemplates that the “C-terminus of a peptide may consist of an amino acid sequence,” which refers to the C-terminus of the peptide having the exact number of amino acids in the sequence and not more or having not more than a rage of amino acids specified in the claim however the N-terminus may be connected to additional amino acids, e.g., as part of a larger peptide.

In certain embodiments, the disclosure relates to compositions comprising chimeric proteins, nucleic acids, vectors, or particles disclosed herein and a pharmaceutically acceptable excipient. In certain embodiments, the disclosure relates to immunogenic compositions comprising chimeric proteins, nucleic acids, vectors, or particles disclosed herein.

In certain embodiments, the disclosure contemplates delivery devices comprising a composition disclosed herein. In certain embodiments, the delivery device is a needle comprising a hollow housing, wherein the chimeric proteins, vectors, nucleic acids, or particles disclosed herein are contained within the hollow housing. In certain embodiments, the delivery device is a needle comprising a biodegradable polymer or non-biodegradable solid, wherein the chimeric proteins, vectors, nucleic acids, or particles are contained within the biodegradable solid or coated on the needle. In certain embodiments, the delivery device is a needle with a diameter of less than one hundred micrometers or one millimeter or a shaft length of less than one hundred micrometers or one millimeter. In certain embodiments, the microneedle length is 100-700 microns. In certain embodiments, delivery device is a syringe with a needle. In certain embodiments, delivery device is a syringe, and the needle length is between 4-12 mm. In certain embodiments, the needle is configured with an adaptor that prevents the needle from penetrating the skin not more than one millimeter or not more than one hundred micrometers.

As used herein, the terms “pharmaceutically acceptable carrier” and “pharmaceutically acceptable vehicle” are interchangeable and refer to a fluid vehicle for containing vaccine antigens that can be injected into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, dyes, and the like.

As used herein, the term “vaccine composition” includes chimeric proteins, nucleic acids, vectors, or particles disclosed herein in a pharmaceutically acceptable vehicle useful for inducing an immune response in a host. Vaccine compositions can be administered in dosages and by techniques well known to those skilled in the medical or veterinary arts, taking into consideration such factors as the age, sex, weight, species and condition of the recipient animal, and the route of administration. The route of administration can be percutaneous, via mucosal administration (e.g., oral, nasal, anal, vaginal) or via a parenteral route (intradermal, intramuscular, subcutaneous, intravenous, or intraperitoneal). Vaccine compositions can be administered alone or can be co-administered or sequentially administered with other treatments or therapies. Forms of administration may include suspensions, syrups or elixirs, and preparations for parenteral, subcutaneous, intradermal, intramuscular, or intravenous administration (e.g., injectable administration) such as sterile suspensions or emulsions. Vaccine compositions may be administered as a spray or mixed in food and/or water or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling, or viscosity enhancing additives, preservatives, flavoring agents, colors, and the like, depending upon the route of administration and the preparation desired. Standard pharmaceutical texts, such as “Remington's Pharmaceutical Sciences,” 1990 may be consulted to prepare suitable preparations, without undue experimentation.

As used herein, the term “immune response” refers to a response elicited in an animal. An immune response may refer to cellular immunity (CMI), humoral immunity or may involve both.

As used herein, the term “adjuvant” means a substance added to a vaccine to increase immunogenicity of a vaccine. Some adjuvants are believed to enhance the immune response by slowly releasing the antigen, while other adjuvants are strongly immunogenic in their own right and are believed to function synergistically.

An “effective amount” of an immunogenic composition, e.g. as used in a vaccine of the disclosure refers to an amount sufficient to show a meaningful benefit in a subject being treated, when administered as part of a vaccination dosing regimen. Those of ordinary skill in the art will appreciate that, in some embodiments, a particular composition may be considered to contain a prophylactically or therapeutically effective amount if it contains an amount appropriate for a unit dosage form administered in a specific dosing regimen, even though such amount may be insufficient to achieve the meaningful benefit if administered as a single unit dose. Those of ordinary skill will further appreciate that an effective amount of an immunogenic composition may differ for different subjects receiving the composition, for example depending on such factors as the desired biological endpoint, the nature of the composition, the route of administration, the health, size and/or age of the subject being treated, etc. In some embodiments, an effective amount is one that has been correlated with beneficial effect when administered as part of a particular dosing regimen, e.g. a single administration or a series of administrations such as in a “boosting” regimen.

As used herein, “subject” refers to any animal, preferably a human patient, livestock, or domestic pet.

The term “nucleic acid” refers to a polymer of nucleotides, or a polynucleotide. The term is used to designate a single molecule, or a collection of molecules. Nucleic acids may be single stranded or double stranded and may include coding regions and regions of various control elements, as described below. The term “a polynucleotide having “a nucleic acid sequence encoding” a specified polypeptide refers to a nucleic acid sequence comprising the coding region of a nucleic acid sequence which encodes a protein product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors may contain endogenous enhancers/promoters, splice junctions, intervening sequences, polyadenylation signals, etc. or a combination of both endogenous and exogenous control elements.

The terms “protein” and “polypeptide” refer to compounds comprising amino acids joined via peptide bonds and are used interchangeably. As used herein, where “amino acid sequence” is recited herein to refer to an amino acid sequence of a protein molecule. An “amino acid sequence” can be deduced from the nucleic acid sequence encoding the protein. However, terms such as “polypeptide” or “protein” are not meant to limit the amino acid sequence to the deduced amino acid sequence but include post-translational modifications of the deduced amino acid sequences, such as amino acid deletions, additions, and modifications such as glycosylations and addition of lipid moieties.

The term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences includes those obtained from the same or from different species of organisms.

The term “heterologous polypeptide” refers to polypeptide that contains two polypeptides joined together such that the entire sequence does not exist in a natural environment. For example, a heterologous polypeptide includes a chimeric protein derived from joining a polypeptide sequence form one species with a polypeptide of another species. Heterologous polypeptides may be produced from heterologous nucleic acid sequences, e.g., DNA, cDNA, RNA, etc. DNA sequences may be expressed in either a sense (to produce mRNA) or anti-sense orientation (to produce an anti-sense RNA transcript that is complementary to the mRNA transcript). Heterologous nucleic acids sequences are typically the union of nucleotide sequences comprising regulatory elements such as promoters that are not found naturally associated with the heterologous nucleic acid sequence for the protein encoded by the heterologous nucleic acid sequence.

In certain embodiments, the disclosure relates to the recombinant vectors comprising a nucleic acid encoding a polypeptide disclosed herein or chimeric protein thereof. The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free. Example “vectors” include plasmids, cosmids, chromosomes, phage, virus, and the like, which is capable of replication when associated with proper control elements. Unique restriction enzyme sites can be included at the 5′ and 3′ ends of expression vectors to allow for insertion into a polynucleotide.

Nucleic acid sequences necessary for expression in prokaryotes usually include a promoter, an operator (optional), and a ribosome-binding site, often along with other sequences. Eukaryotic cells are known to utilize promoters, enhancers, and termination and polyadenylation signals. For expression in animal cells, an expression vector can comprise suitable promoters that can drive transcription of the polynucleotide sequence. If the cells are mammalian cells, then promoters such as, for example, actin promoter, metallothionein promoter, NF-kappaB promoter, EGR promoter, SRE promoter, IL-2 promoter, NFAT promoter, osteocalcin promoter, SV40 early promoter and SV40 late promoter, Lck promoter, BMPS promoter, TRP-1 promoter, murine mammary tumor virus long terminal repeat promoter, STAT promoter, or an immunoglobulin promoter can be used in the expression vector.

In certain embodiments, the vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

Vectors often contain a selectable marker or screenable marker. A “selectable marker” is a nucleic acid introduced into a recombinant vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme that confers a blue color in the presence of X-gal (5-bromo-4-chloro-3-indolyl-(3-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: amp^{r}, cam^{r}, tet^{r}, blasticidin^{r}, neo^{r}, hyg^{r}, abx^{r}, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (at1D), UDP-glucose:galactose-1-phosphate uridyltransferasel (galT), feedback-insensitive a subunit of anthranilate synthase (OASAID), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

Protein “expression systems” refer to in vivo and in vitro (cell free) systems. Systems for recombinant protein expression typically utilize cells (somatic cells) transfected with a DNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation, and optionally post-translational modifications such as RNA polymerase, regulatory protein factors, transcription factors, ribosomes, tRNA cofactors, amino acids, and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

In certain embodiments, the disclosure relates to chimeric polypeptides comprising sequences disclosed herein, or variants or fusions thereof wherein the amino terminal end or the carbon terminal end of the amino acid sequence are optionally attached to a heterologous amino acid sequence, label, or reporter molecule.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous polypeptide in the receptor. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

In certain embodiments, this disclosure contemplates that fusion or chimeric proteins disclosed herein may be variants. Variants may include 1 or 2 amino acid substitutions or conserved substitutions. Variants may include 3 or 4 amino acid substitutions or conserved substitutions. Variants may include 5 or 6 or more amino acid substitutions or conserved substitutions. Variants include those with not more than 1% or 2% of the amino acids are substituted. Variants include those with not more than 3% or 4% of the amino acids are substituted. Variants include proteins with greater than 80%, 89%, 90%, 95%, 98%, or 99% identity or similarity.

Variants can be tested by mutating the vector to produce appropriate codon alternatives for polypeptide translation. Active variants and fragments can be identified with a high probability using computer modeling. Shihab et al. report an online genome tolerance browser. BMC Bioinformatics. 2017, 18(1):20. Ng et al. report methods of predicting the effects of amino acid substitutions on protein function. Annu Rev Genomics Hum Genet. 2006, 7:61-80. Teng et al. Approaches and resources for prediction of the effects of non-synonymous single nucleotide polymorphism on protein function and interactions. Curr Pharm Biotechnol. 2008, 9(2):123-33.

Guidance in determining which and how many amino acid residues may be substituted, inserted, or deleted without abolishing biological activity may be found using computer programs well known in the art, for example, RaptorX, ESyPred3D, HHpred, Homology Modeling Professional for HyperChem, DNAStar, SPARKS-X, EVfold, Phyre, and Phyre2 software. See also Saldano et al. Evolutionary Conserved Positions Define Protein Conformational Diversity, PLoS Comput Biol. 2016, 12(3):e1004775; Marks et al. Protein structure from sequence variation, Nat Biotechnol. 2012, 30(11):1072-80; Mackenzie et al. Curr Opin Struct Biol. 2017, 44:161-167 Mackenzie et al. Proc Natl Acad Sci USA. 113(47):E7438-E7447 (2016); Joseph et al. J R Soc Interface. 2014, 11(95):20131147, Wei et al. Int. J. Mol. Sci. 2016, 17(12), 2118. Variants can be tested in functional assays. Certain variants have less than 10%, and preferably less than 5%, and still more preferably less than 2% changes (whether substitutions, deletions, and so on).

Polynucleotides, vectors, and expression constructs can be introduced in vivo via lipofection (DNA transfection via liposomes prepared from synthetic cationic lipids). Synthetic cationic lipids can be used to prepare liposomes to encapsulate a polynucleotide, vector, or expression construct of the invention. A polynucleotide, vector, or expression construct can also be introduced as naked DNA or RNA using methods known in the art, such as transfection, microinjection, electroporation, calcium phosphate precipitation, and by biolistic methods.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q. The amino acid groups are also considered conserved substitutions.

It is contemplated that proteins disclosed herein may contain linkers. The purpose of a linker is to allow the correct formation, folding and/or functioning of each of a chimeric protein. It should be sufficiently flexible and sufficiently long to achieve that purpose. Typically, the coding sequence of the linker may be chosen such that it encourages translational pausing and therefore independent folding of the protein. A person skilled in the art will be able to design suitable linkers in accordance with the disclosure. Multiple copies of the linker sequence of choice may be inserted between polypeptide segments. The only requirement for the linker sequence is that it functionally does not adversely interfere with the folding and/or functioning of the individual entities of the fusion protein. For example, a suitable linker may be 1 to 5 or 5 to 50 amino acid long and may comprise amino acids such as glycine, serine, threonine, asparagine, alanine and proline (see for example Wiederrecht et al., 1988, Cell 54, 841; Dekker et al., 1993, Nature 362, 852; Sturm et al., 1988, Genes and Dev. 2, 1582; Aumailly et al., 1990 FEBS Lett. 262, 82). Repeats comprising serine and glycine residues are typical in the context of the disclosure. It will be evident that, in certain embodiments, the disclosure is not limited to the use of these particular linkers.

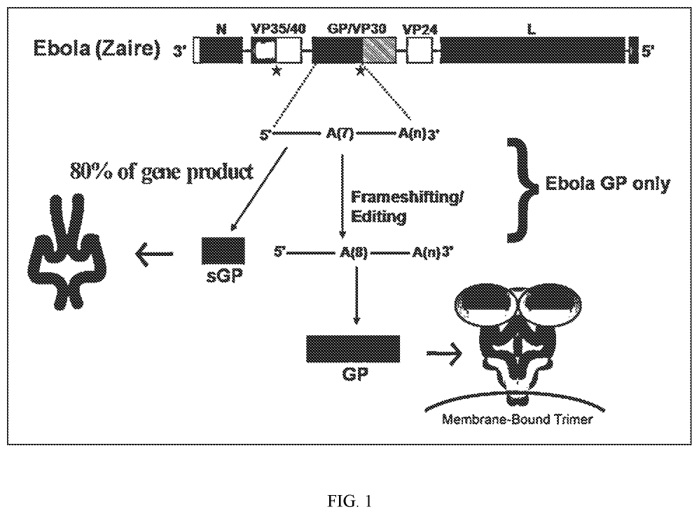

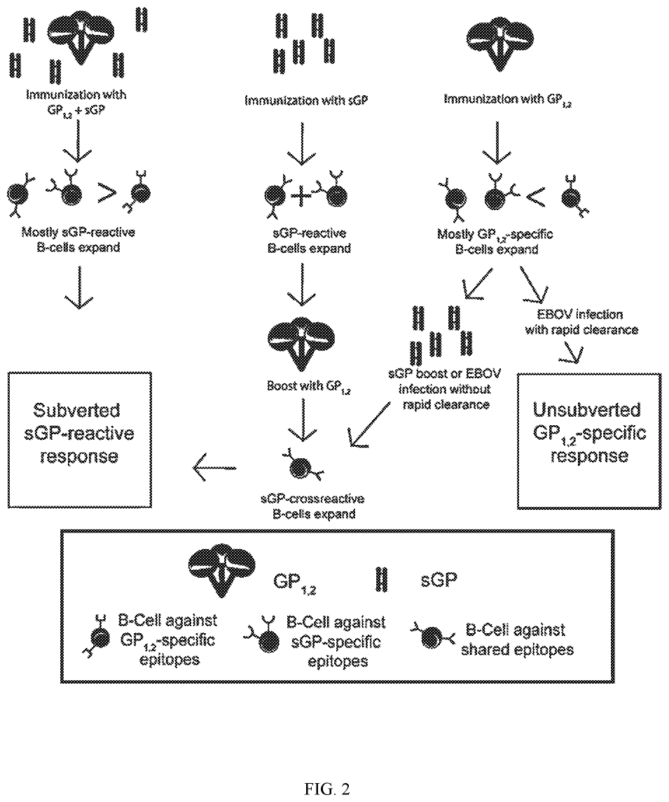

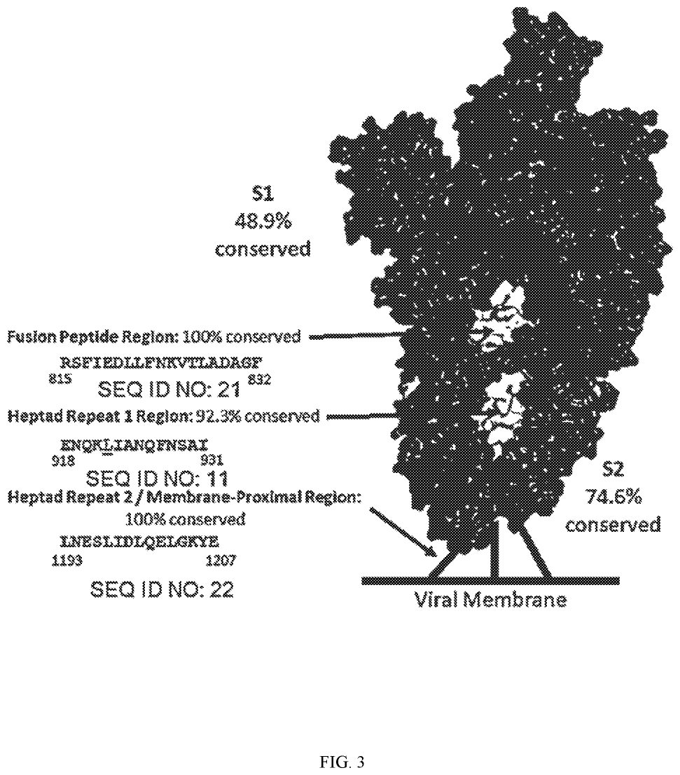

As shown in the FIG. 1, the Ebola virus (EBOV) sGP is synthesized as a single polypeptide forms a dimer structure after synthesis. It is the major glycoprotein product during virus infection, and it is efficiently secreted from cells after synthesis.

This disclosure relates to scaffolds for inducing antibody responses against antigenic sites. In certain embodiments, this disclosure relates to compositions and methods using a filovirus sGP as a scaffold for inducing antibody responses against antigenic sites in foreign pathogens. In certain embodiments, this disclosure relates to compositions and methods using a filovirus sGP as a scaffold for inducing antibody responses against viruses, e.g., coronavirus to produce a coronavirus, e.g., SARS-Cov-2, vaccine, or other viral vaccine.

In certain embodiments, this disclosure relates to fusion proteins, or vectors encoding the same, comprising a heterologous sequence inserted in the middle of a filovirus soluble glycoprotein (sGP). In certain embodiments, the filovirus is selected form Ebola virus (EBOV), Sudan virus (SUDV); Bundibugyo virus (BDBV); Tai Forest virus (TAFV); Reston virus (RESTV); Marburg virus (MARV); and Lloviu virus (LLOV). In certain embodiments, the heterologous sequence is a microbial sequence, viral sequence or coronavirus sequence.

In certain embodiments, the fusion protein has a linker comprising glycine and/or serine between a filovirus sequence and the heterologous sequence or coronavirus sequence.

In certain embodiments, this disclosure relates to fusion proteins, nucleic acids, or vectors encoding the same, wherein the fusion protein comprises or consists of the amino acids sequence of
MGVTGILQLPRDRFKRTSFFLWVIILFQRTFSIPLGVIHNSTLQVSDVDKLVCRDK LSSTNQLRSVGLNLEGNGVATDVPSATKRWGFRSGVPPKVVNYEAGEWAENCYNLEIK KPDGSECLPAAPDGIRGFPRCRYVHKVSGTGPCAGDFAFHKEGAFFLYDRLASTVIYRG TTFAEGVVAFLILPQAKKDFFSSHPLREPVNATEDPSSGYYSTTIRYQATGFGTNETEYLF EVDNLTYVQLESRFTPQFLLQLNETIYTSGKRSNTTGKLIWKVNPEIDTTIGERSFIEDLLF NKVTLADAGKFAVKSCLSQLYQTEPKTSVV (SEQ ID NO: 1) (Group 1: FP-end) or variant thereof.

In certain embodiments, the heterologous sequence is a coronavirus sequence inserted between amino acids corresponding to amino acids 287 and 300 of Ebola sGP, such as MGVTGILQLPRDRFKRTSFFLWVIILFQRTFSIPLGVIHNSTLQVSDVDKLVCRDKLSSTN QLRSVGLNLEGNGVATDVPSATKRWGFRSGVPPKVVNYEAGEWAENCYNLEIKKPDG SECLPAAPDGIRGFPRCRYVHKVSGTGPCAGDFAFHKEGAFFLYDRLASTVIYRGTTFAE GVVAFLILPQAKKDFFSSHPLREPVNATEDPSSGYYSTTIRYQATGFGTNETEYLFEVDN LTYVQLESRFTPQFLLQLNETIYTSGKRSNTTGKLIWKVNPEIDTTIGERSFIEDLLFNKV TLADAGKFAVKSCLSQLYQTEPKTSVV (SEQ ID NO: 1)(Group 1: FP-end), wherein DTTIGE (SEQ ID NO: 2) are amino acid positions 281 to 287 of SEQ ID NO: 1 and KFAVKS (SEQ ID NO: 3) are amino acid positions 300 to 395 of SEQ ID NO: 1. In certain embodiments, the coronavirus sequence is RSFIEDLLFNKVTLADAG (SEQ ID NO: 4) or variant thereof.

In certain embodiments, this disclosure relates to fusion proteins, nucleic acids, or vectors encoding the same, wherein the fusion protein comprises or consists of the amino acid sequence of
MGVTGILQLPRDRFKRTSFFLWVIILFQRTFSIPLGVIHNSTLQVSDVDKLVCRDK LSSTNQLRSVGLNLEGNGVATDVPSATKRWGFRSGVPPKVVNYEAGEWAENCYNLEIK KPDGSECLPAAPDGIRGFPRCRYVHKVSGTGPCAGDFAFHKEGAFFLYDRLASTVIYRG TTFAEGVVAFLILPSKRSFIEDLLFNKVTLADAGPSSGYYSTTIRYQATGFGTNETEYLFE VDNLTYVQLESRFTPQFLLQLNETIYTSGKRSNTTGKLIWKVNPEIDTTIGEWAFWETKK TSLEKFAVKSCLSQLYQTEPKTSVV (SEQ ID NO: 5) (Group 2: FP-mid) or variant thereof.

In certain embodiments, the heterologous sequence is a coronavirus sequence inserted between amino acids corresponding to amino acids 187 and 209 of Ebola sGP, such as, MGVTGILQLPRDRFKRTSFFLWVIILFQRTFSIPLGVIHNSTLQVSDVDKLVCRDKLSSTN QLRSVGLNLEGNGVATDVPSATKRWGFRSGVPPKVVNYEAGEWAENCYNLEIKKPDG SECLPAAPDGIRGFPRCRYVHKVSGTGPCAGDFAFHKEGAFFLYDRLASTVIYRGTTFAE GVVAFLILPSKRSFIEDLLFNKVTLADAGPSSGYYSTTIRYQATGFGTNETEYLFEVDNL TYVQLESRFTPQFLLQLNETIYTSGKRSNTTGKLIWKVNPEIDTTIGEWAFWETKKTSLE KFAVKSCLSQLYQTEPKTSVV (SEQ ID NO: 5) (Group 2: FP-mid), wherein AFLILP (SEQ ID NO: 6) are amino acid positions 181 to 187 of SEQ ID NO: 5 and PSSGYY (SEQ ID NO: 7) are amino acid positions 209 to 214. In certain embodiments, the coronavirus sequence is SKRSFIEDLLFNKVTLADAG (SEQ ID NO: 8) or variant thereof.

In certain embodiments, this disclosure relates to fusion proteins, nucleic acids, or vectors encoding the same, wherein the fusion protein comprises or consists of the amino acid sequence of
MGVTGILQLPRDRFKRTSFFLWVIILFQRTFSIPLGVIHNSTLQVSDVDKLVCRDK LSSTNQLRSVGLNLEGNGVATDVPSATKRWGFRSGVPPKVVNYEAGEWAENCYNLEIK KPDGSECLPAAPDGIRGFPRCRYVHKVSGTGPCAGDFAFHKEGAFFLYDRLASTVIYRG TTFAEGVVAFLILPENQKLIANQFNSAIATEDPSSGYYSTTIRYQATGFGTNETEYLFEVD NLTYVQLESRFTPQFLLQLNETIYTSGKRSNTTGKLIWKVNPEIDTTIGERSFIEDLLFNKV TLADAGKFAVKSCLSQLYQTEPKTSVV (SEQ ID NO: 9) (Group 3: HR1/FP) or variant thereof.

In certain embodiments, the heterologous sequence is a first coronavirus sequence inserted between amino acids corresponding to amino acids 187 and 205 and second coronavirus sequence inserted between the amino acids corresponding to amino acids 287 and 300 of Ebola sGP, such as
MGVTGILQLPRDRFKRTSFFLWVIILFQRTFSIPLGVIHNSTLQVSDVDKLVCRDKLSSTN QLRSVGLNLEGNGVATDVPSATKRWGFRSGVPPKVVNYEAGEWAENCYNLEIKKPDG SECLPAAPDGIRGFPRCRYVHKVSGTGPCAGDFAFHKEGAFFLYDRLASTVIYRGTTFAE GVVAFLILPENQKLIANQFNSAIATEDPSSGYYSTTIRYQATGFGTNETEYLFEVDNLTY VQLESRFTPQFLLQLNETIYTSGKRSNTTGKLIWKVNPEIDTTIGERSFIEDLLFNKVTLA DAGKFAVKSCLSQLYQTEPKTSVV (SEQ ID NO: 9) (Group3: FP-mid), wherein AFLILP (SEQ ID NO: 6) are amino acid positions 181 to 187 of SEQ ID NO: 9 and ATEDPS (SEQ ID NO: 10) are amino acid positions 205 to 210, and wherein DTTIGE (SEQ ID NO: 2) are amino acid positions 281 to 287 and KFAVKS (SEQ ID NO: 3) are amino acid positions 300 to 305. In certain embodiments, the first coronavirus sequence is ENQKLIANQFNSAI (SEQ ID NO: 11) or variants thereof, and the second coronavirus sequence is RSFIEDLLFNKVTLADAG (SEQ ID NO: 4) or variant thereof.

In certain embodiments, this disclosure relates to fusion proteins, nucleic acids, or vectors encoding the same, wherein the fusion protein comprises or consists of the amino acid sequence of
MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL ASKRSFIEDLLFNKVTLADAGTSSYYATSYLEYEIENFGAQHSTTLFKIDNNTFVRLDRPH TPQFLFQLNDTIHLHQQLSNTTGRLIWTLDANINADIGERSFIEDLLFNKVTLADAGKFAV KSCLSQLYQTEPKTSVV (SEQ ID NO: 12)(SUDV FP/FP) or variants thereof.

In certain embodiments, the heterologous sequence is Sudan viruses with an N-terminal melittin signal sequence MKFLVNVALVF (SEQ ID NO: 13), a first a coronavirus sequence inserted between amino acids corresponding to amino acids 187 and 209 of a Sudan sGP and second coronavirus sequence inserted between the amino acids corresponding to amino acids 287 and 300 of Sudan virus sGP and containing a C-terminal Ebola sequence, such as, MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL ASKRSFIEDLLFNKVTLADAGTSSYYATSYLEYEIENFGAQHSTTLFKIDNNTFVRLDRP HTPQFLFQLNDTIHLHQQLSNTTGRLIWTLDANINADIGERSFIEDLLFNKVTLADAGKF AVKSCLSQLYQTEPKTSVV (SEQ ID NO: 12)(SUDV FP/FP)), wherein AFLILA (SEQ ID NO: 30) are amino acid positions 181 to 187 of SEQ ID NO: 12 and TSSYYA (SEQ ID NO: 14) are amino acid positions 209 to 214, and wherein NADIGE (SEQ ID NO: 15) are amino acid positions 281 to 287 and KFAVKS (SEQ ID NO: 3) are amino acid positions 300 to 305, and the C-terminal Ebola sequence is KFAVKSCLSQLYQTEPKTSVV (SEQ ID NO: 16).

In certain embodiments, this disclosure relates to fusion proteins, nucleic acids, or vectors encoding the same, wherein the fusion protein comprises or consists of the amino acid sequence of
MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL AKPKETFLQSPPIREAVNYTENTSSYYATSYLEYEIENFGAQHSTTLFKIDNNTFVRLDRP HTPQFLFQLNDTIHLHQQLSNTTGRLIWTLDANINADIGERSFIEDLLFNKVTLADAGNY VEKSCLSKLYRSTRQKTMMR (SEQ ID NO: 17)(Grp 1: SUDV FP-end) or variants thereof.

In certain embodiments, the heterologous sequence is Sudan viruses with an N-terminal melittin signal sequence MKFLVNVALVF (SEQ ID NO: 13), and a coronavirus sequence inserted between the amino acids corresponding to amino acids 287 and 300 of Sudan virus sGP, such as MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL AKPKETFLQSPPIREAVNYTENTSSYYATSYLEYEIENFGAQHSTTLFKIDNNTFVRLDRP HTPQFLFQLNDTIHLHQQLSNTTGRLIWTLDANINADIGERSFIEDLLFNKVTLADAGN YVEKSCLSKLYRSTRQKTMMR (SEQ ID NO: 17)(Grp 1: SUDV FP-end), wherein NADIGE (SEQ ID NO: 15) are amino acid positions 281 to 287 and NYVEKS (SEQ ID NO: 18) are amino acid positions 300 to 305.

In certain embodiments, this disclosure relates to fusion proteins, nucleic acids, or vectors encoding the same, wherein the fusion protein comprises or consists of the amino acid sequence of
MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL ASKRSFIEDLLFNKVTLADAGTSSYYATSYLEYEIENFGAQHSTTLFKIDNNTFVRLDRPH TPQFLFQLNDTIHLHQQLSNTTGRLIWTLDANINADIGERSFIEDLLFNKVTLADAGNYV EKSCLSKLYRSTRQKTMMR (SEQ ID NO: 19)(Grp 2: SUDV FP/FP) or variants thereof.

In certain embodiments, the heterologous sequence is Sudan viruses with an N-terminal melittin signal sequence MKFLVNVALVF (SEQ ID NO: 13), a first a coronavirus sequence inserted between amino acids corresponding to amino acids 187 and 208 of a Sudan sGP and second coronavirus sequence inserted between the amino acids corresponding to amino acids 287 and 300 of Sudan virus sGP, such as,
MKFLVNVALVFMVVYISYIYAAMPLGVVTNSTLEVTEIDQLVCKDHLASTDQL KSVGLNLEGSGVSTDIPSATKRWGFRSGVPPKVVSYEAGEWAENCYNLEIKKPDGSECL PPPPDGVRGFPRCRYVHKAQGTGPCPGDYAFHKDGAFFLYDRLASTVIYRGVNFAEGVI AFLILASKRSFIEDLLFNKVTLADAGTSSYYATSYLEYEIENFGAQHSTTLFKIDNNTFV RLDRPHTPQFLFQLNDTIHLHQQLSNTTGRLIWTLDANINADIGERSFIEDLLFNKVTLA DAGNYVEKSCLSKLYRSTRQKTMMR (SEQ ID NO: 19)(Grp 2: SUDV FP/FP), wherein AFLILA (SEQ ID NO: 30) are amino acid positions 181 to 187 of SEQ ID NO: 19 and TSSYYA (SEQ ID NO: 14) are amino acid positions 208 to 213, and wherein NADIGE (SEQ ID NO: 15) are amino acid positions 281 to 287 and NYVEKS (SEQ ID NO: 18) are amino acid positions 300 to 305.

In certain embodiments, this disclosure relates to fusion proteins, nucleic acids, or vectors encoding the same, wherein the fusion protein comprises or consists of the amino acid sequence of
MKFLVNVALVFMVVYISYIYAAMPLGVVTNSTLEVTEIDQLVCKDHLASTDQLKSVGL NLEGSGVSTDIPSATKRWGFRSGVPPKVVSYEAGEWAENCYNLEIKKPDGSECLPPPPD GVRGFPRCRYVHKAQGTGPCPGDYAFHKDGAFFLYDRLASTVIYRGVNFAEGVIAFLIL ASKRSFIEDLLFNKVTLADAGTSSYYATSYLEYEIENFGAQHSTTLFKIDNNTFVRLDRPH TPQFLFQLNDTIHLHQQLSNTTGRLIWTLDANINADIGEWAFWENKKISPNNYVEKSCLS KLYRSTRQKTMMR (SEQ ID NO: 20)(Grp 3:SUDV FP-mid) or variants thereof.

In certain embodiments, the heterologous sequence is Sudan viruses with an N-terminal melittin signal sequence MKFLVNVALVF (SEQ ID NO: 13), a first a coronavirus sequence inserted between amino acids corresponding to amino acids 187 and 208 of a Sudan sGP, such as,
MKFLVNVALVFMVVYISYIYAAMPLGVVTNSTLEVTEIDQLVCKDHLASTDQL KSVGLNLEGSGVSTDIPSATKRWGFRSGVPPKVVSYEAGEWAENCYNLEIKKPDGSECL PPPPDGVRGFPRCRYVHKAQGTGPCPGDYAFHKDGAFFLYDRLASTVIYRGVNFAEGVI AFLILASKRSFIEDLLFNKVTLADAGTSSYYATSYLEYEIENFGAQHSTTLFKIDNNTFV RLDRPHTPQFLFQLNDTIHLHQQLSNTTGRLIWTLDANINADIGEWAFWENKKISPNNYV EKSCLSKLYRSTRQKTMMR (SEQ ID NO: 20)(Grp 3: SUDV FP-mid), wherein AFLILA (SEQ ID NO: 30) are amino acid positions 181 to 187 of SEQ ID NO: 19 and TSSYYA (SEQ ID NO: 14) are amino acid positions 208 to 213.

In certain embodiments, this disclosure relates to fusion proteins, nucleic acids, or vectors encoding the same, wherein the coronavirus sequence comprises an amino acid sequence selected from RSFIEDLLFNKVTLADAG (SEQ ID NO: 4), ENQKLIANQFNSAI (SEQ ID NO: 11), and LNESLIDLQELGKYE (SEQ ID NO: 22), or variants with greater than 60%, 70%, 80%, or 90% identity.

In certain embodiments, this disclosure relates to nucleic acid encoding a fusion protein disclosed herein in operable combination with a promoter/heterologous promoter, wherein the fusion protein comprise a heterologous sequence inserted in the middle of filovirus soluble glycoprotein (sGP). In certain embodiments, this disclosure relates to a vector comprising a nucleic acid encoding a fusion protein disclosed herein. In certain embodiments, the nucleic acid or vector is DNA or RNA.

In certain embodiments, the variant of a fusion protein disclosed herein has greater than 70% 80%, 90%, 95%, 96%, 97%, 98%, 99%, identity to a sequence disclosed herein.

In certain embodiments, this disclosure relates to virus particles or virus like particles comprising a fusion protein comprising a heterologous or coronavirus sequence as disclosed herein, e.g., inserted between amino acids corresponding to amino acids 187 and 213 of Ebola sGP.

In certain embodiments, this disclosure relates to expression system comprising a vector or nucleic acid encoding a fusion protein in operable combination with a promoter, wherein the fusion protein comprising a heterologous or coronavirus sequence as disclosed herein, e.g., inserted in the middle of filovirus soluble glycoprotein (sGP) corresponding to amino acids 187 and 213 of sGP of Ebola.

In certain embodiments, this disclosure relates to methods of treating or preventing a viral infection or coronavirus infection or reducing the symptoms of a viral or coronavirus infection comprising administering to a subject in need thereof an effective amount of a fusion protein as provided disclosed herein optionally in combination with an adjuvant.

In certain embodiments, this disclosure relates to methods of treating or preventing a coronavirus infection or reducing the symptoms of a viral infection or coronavirus infection comprising administering to a subject in need thereof an effective amount of a nucleic acid or vector encoding a fusion protein in operable combination with a promoter, wherein the fusion protein is as disclosed herein optionally the nucleic acid or vector is administered in combination with an adjuvant. In certain embodiments the nucleic acid is DNA or RNA

In certain embodiments, this disclosure relates to methods of treating or preventing a viral infection or coronavirus infection or reducing the symptoms of a viral infection or coronavirus infection comprising administering to a subject in need thereof an effective amount of a virus particle or virus like particle comprising a fusion protein as described herein.

### Filovirus Glycoprotein sGP as a Scaffold to Present Non-Filovirus Antigens for Vaccination

Vaccine development against infectious pathogens (such as virus and bacteria) is of paramount importance for public health. However, in many cases, the antigen itself may not be optimal for vaccine production or for eliciting desired immune responses, e.g., due to its antigenic properties, stability, as well as shielding of important antigenic site from immune system. Although the coronavirus has a proofreading mechanism during replication, mutations do occur, particularly in its spike glycoprotein S, that sometimes enables the virus to escape the host immune response. For examples, a single amino acid change in the receptor binding domain (RBD) of SARS-COV S protein was found in infected humans and such a change led to virus escape from a potently neutralizing antibodies.

As shown in Table 1, the S1 subunit of the SARS-COV-2 S protein shared approximately 50% of its amino acid sequence with the S1 subunit of the SARS-COV S protein as well as to the S protein of other lineage B β-coronavirus. On the other hand, the S2 subunit was found to exhibit approximately 75% sequence homology. Three segments were identified in the S2 subunit, designated as the Fusion Peptide Region (FP, amino acid 815-832 in the SARS-COV-2 S), the Heptad Repeat 1 Region (HR1, amino acid 918-931 in the SARS-COV-2 S), and the Heptad Repeat 2/Membrane-Proximal Region (HR2/MPR, amino acid 1193-1207 in the SARS-COV-2 S) are highly conserved among all lineage B f3-coronavirus, with 92-100% in amino acid sequence homology.

TABLE 1
Amino acid (AA) identity in different regions of the S
protein between lineage B β-coronaviruses

| Region | Percent Homology |
|---|---|
| Full S Protein | 66.6% |
| S1 Subunit | 48.9% |
| S2 Subunit | 74.6% |
| FP (AA 815-832) | 1 00% |
| HR1 (AA 918-931) | 92.3% |
| HR2/MPR (AA 1193-1207) | 100% |

The conserved antigenic sites (epitopes) in the human immunodeficiency virus (HIV) envelope glycoprotein (Env) for broadly neutralizing antibodies are heavily shielded by variable domains in the HIV Env, and these variable domains are often highly immune dominant. As a result, inducing broadly neutralizing antibodies against HIV to these conserved epitopes are very difficult to induce by vaccination. Therefore, the development of vaccine strategies by using a scaffold protein to present such conserved epitopes more effectively to the immune system is of high interest. Similarly, such an approach (designing vaccine by presenting an antigen on a scaffold) also represents a highly attractive strategy to develop vaccines that can induce desired immune responses against a viral, bacterial, to confer more effective control against infection or disease progression.

The results presented in above studies show that antigenic fragments from the foreign antigens (the fusion peptide of the SARS-COV2 virus) can be inserted into the EBOV (Ebola virus) sGP glycoprotein replacing an exposed segment of the EBOV sGP for expression of chimeric proteins. More importantly, such chimeric proteins are: 1) expressed and secreted; 2) retain dimer formation; 3) retain reactivity to antibodies against EBOV GP; 4) and present the foreign antigen more prominently to the host immune system for induction of antibodies that target to the antigenic epitopes located within these antigens.

Implications of these Findings for Vaccine Development Against Infectious Pathogens:

The filovirus sGP is useful as a scaffold to present a foreign sequence (i.e. an antigenic site from a different protein which could be a viral protein or a protein from a parasite or bacteria, or a cancer antigen), and thereby to induce specific antibody responses against this foreign antigen with the aim to protect vaccinated subjects from subsequent infections by the foreign agent (i.e. use as a vaccine for a virus, parasite, or bacteria, or suppression of tumor growth).

That one can insert the FP (fusion peptide), e.g., HR1 (heptad repeat region 1) of the SARS-COV-2 Spike (S) protein, is evidence that these foreign sequences can be successfully inserted into the sGP at the selected sites (between amino acids 188 and 213 or after amino acid 300 in the Ebola virus sGP) without affecting the sGP production and secretion. The resulting chimeric protein is capable of inducing antibody responses against the inserted foreign sequence, and furthermore such antibodies can react with the foreign antigen from which the inserted sequence was derived. The use of SARS-COV-2 S protein FP or HR1 are examples indicating one could use any sequence from any protein in order to induce an antibody response against the protein (which could be a viral protein, or a protein from a parasite or bacteria or a tumor antigen). EBOV sGP as well as the sGP of other filoviruses are useful as a scaffold to present foreign antigens for vaccine development having following implications and potential utilities for vaccine development against antigens from a wide range of infectious pathogens or host proteins that could be target for vaccine development.

Ebola virus (EBOV) belongs to the filoviridae family, which contained several identified members including EBOV, SUDV, BUDV, TIFV, RSTV, MARV, and LLOV. Some of these viruses (EBOV, SUDV, BUDV, TIFV, and LLOV) express a glycoprotein sGP that is structurally similar to EBOV sGP but antigenically distinct from each other. Thus, by similar approaches, the foreign antigens can be inserted into the antigenically distinct sGP proteins from these different viruses for vaccine development, which will allow for selective boosting specific immune responses against the desired epitopes of in the foreign antigens

The sequence from the SARS-COV2 spike protein (S) inserted into the chimeric proteins retained its immunogenicity to induce antibodies that also recognize the SARS-COV2 Spike Protein (S). Thus, by similar approaches, it can be expected that other non-filovirus antigens such as glycoproteins or portions of glycoproteins from the HIV, Lassa fever viruses, Rift Valley fever viruses, Zika viruses, Dengue viruses, influenza viruses, as well as other viruses, and also surface antigens from bacteria or parasite (for example the Plasmodium that causes Malaria) can be inserted into filovirus sGP at the selected domain region for developing vaccines against these pathogens.

The chimeric proteins can be produced by an expression vector in mammalian cells or insect cells or yeasts and purified in the forms of purified proteins.

The chimeric proteins can be expressed by a recombinant expression vector, such as adenovirus, vaccinia virus, VSV, human parainfluenza virus, rabies virus, as well as a DNA plasmid expression vector, and these vectors can be used in vaccination directly.

This vaccine strategy can be applied to vaccinated humans as well as animals of economic interest against infectious pathogens.

Expression of the sGP-FP Chimeric Protein.

To investigate whether the sGP-based chimeric proteins could be produced in cells, a prototype chimeric protein was constructed, in which the FP of the SARS-COV-2 S protein was inserted into the EBOV sGP, replacing the amino acids 188-213 in the EBOV sGP. The resulting chimeric protein was designated as sGP-FP. After construction, the chimeric protein was expressed in 293T cells by DNA transfection in comparison with the wild type EBOV sGP. The sGP-FP protein was successfully produced in cells and efficiently secreted into cell supernatant similar to the wild type EBOV sGP protein. These results demonstrate that the amino acid 188-213 segment of the sGP can be replaced by a foreign peptide, without affecting the production and secretion of the chimeric protein.

Immunogenicity of the sGP-FP Chimeric Protein.

Experiments were performed to determine whether immunization with the sGP-FP chimeric protein vaccine would induce antibodies against the FP of the SARS-COV-2 S protein.

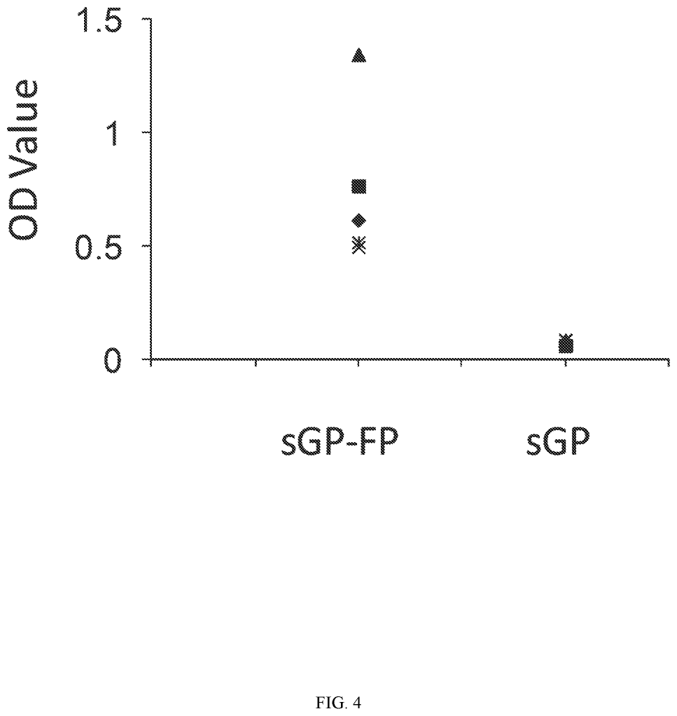

As shown in FIG. 4, immunization with the sGP-FP chimeric protein DNA vaccine successfully induced antibodies that bind to the fusion peptide of the SARS-COV-2 S protein. In contrast, such antibodies were not induced by immunization with the wild-type EBOV sGP DNA vaccine. These results showed that the foreign peptide segment presented on the EBOV sGP scaffold is immunogenic for inducing antibody responses.

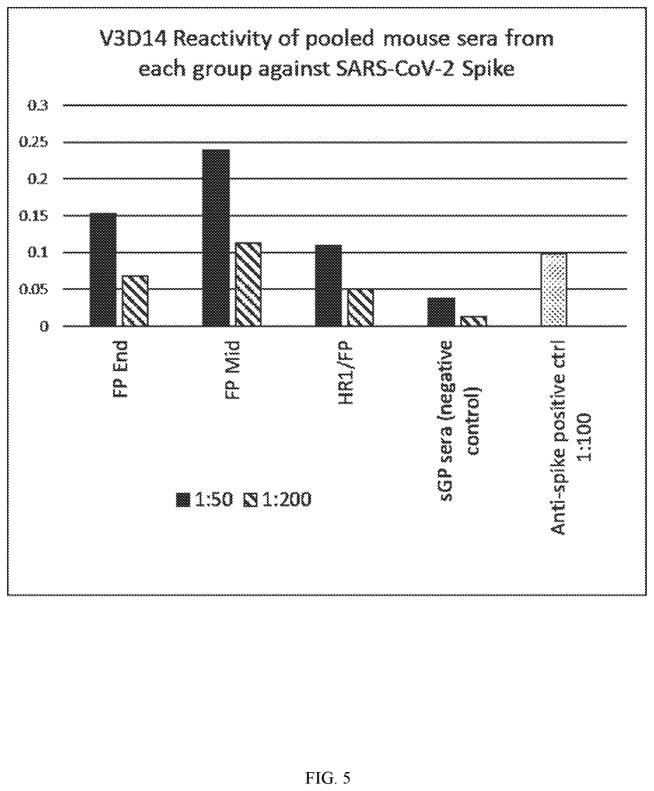

After demonstrating the reactivity of the antibody responses induced by the sGP-FP chimeric protein to the Fusion-peptide (FP), experiments were performed to determine whether such antibodies will also react with the whole SARS-COV2 Spike protein by ELISA using whole SARS-COV2 Spike protein expressed in supernatant of baculovirus-infected-Sf9 cells as a coating antigen. As shown in FIG. 5, the antibodies induced by the sGP-FP chimeric protein (designated as the FP-mid) were found to react to the whole SARS-COV-2 Spike protein. These results further demonstrate the practical utility of this vaccine design strategy to induce functional antibodies for protection against human pathogens.

Boosting of Antibody Response to the SARS-COV-2 FP by a DNA Vaccine Construct in which the FP of SARS-COV-2 Spike Protein was Engrafted into the Same Region of Sudan Virus SGP.

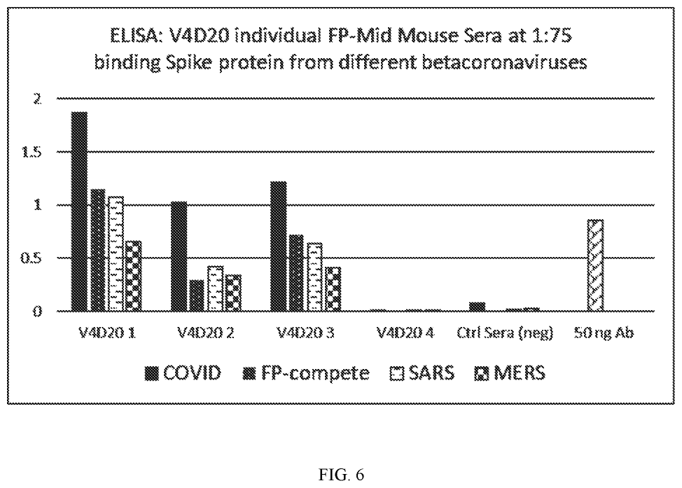

Mice were immunized with a DNA vaccine construct, which expresses a vaccine antigen in which the fusion peptide (FP) of the SARS-CoV-2 Spike protein (amino acids 813-832 in the SARS-COV-2 Spike protein) was inserted into the middle of the Ebola virus soluble glycoprotein (sGP), replacing the amino acids 188-208 in the sGP. The first two doses utilized ebolavirus sGP; the third used Sudan Virus (related virus) sGP with the same amino acids replaced as well as a second copy of the fusion peptide (amino acids 815-832) replacing amino acids 288-299. A fourth vaccination was done with purified protein of the same Sudan Virus sGP construct. ELISA was done on 100 ng/well of pure spike protein from three coronaviruses: SARS-COV-2 (labeled “COVID), SARS-CoV (labeled “SARS), and MERS-CoV (labeled “MERS”). Sera from the fourth vaccination in three out of four mice reacted strongly with spike protein. The “FP-compete” samples were also incubated with a peptide containing fusion peptide residues 813-832 which helped block binding to spike, demonstrating that mouse sera binds COVID spike specifically at the fusion peptide. Samples from the third vaccination and second vaccinations were only tested against COVID spike protein and not the others. Antibody binding at 1:75 exceeds 50 ng of binding for multiple samples, suggestive of a high titer. (FIG. 6)

### Testing Different Constructs:

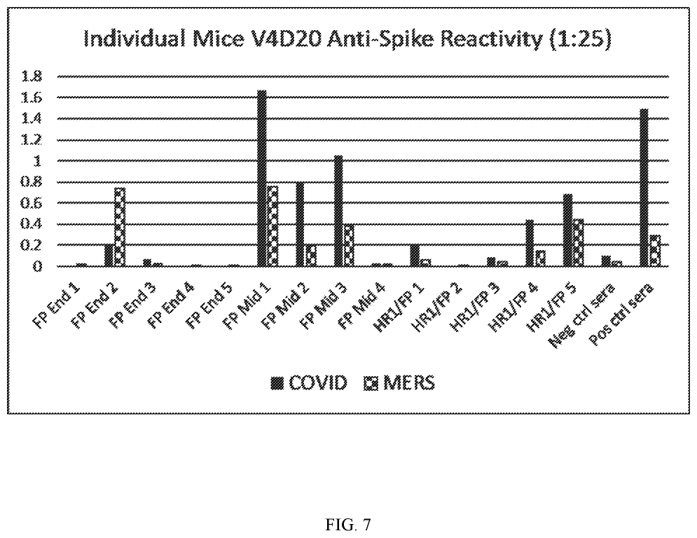

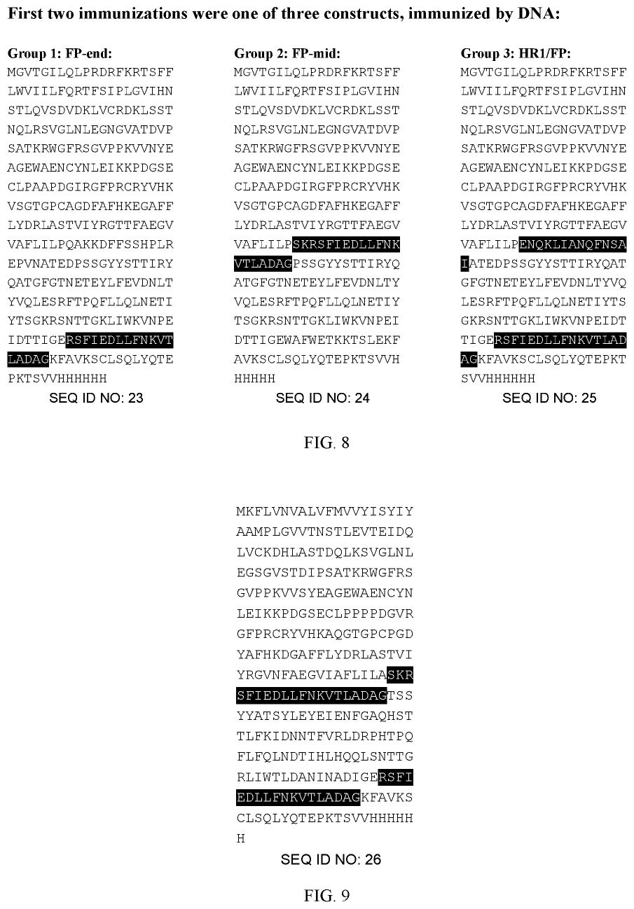

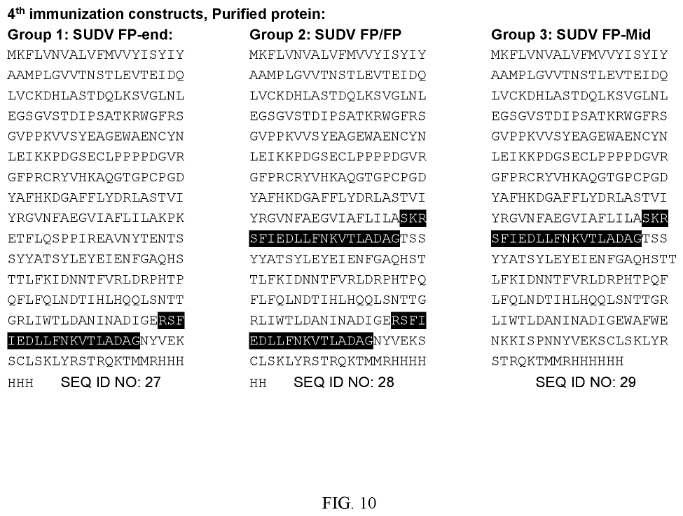

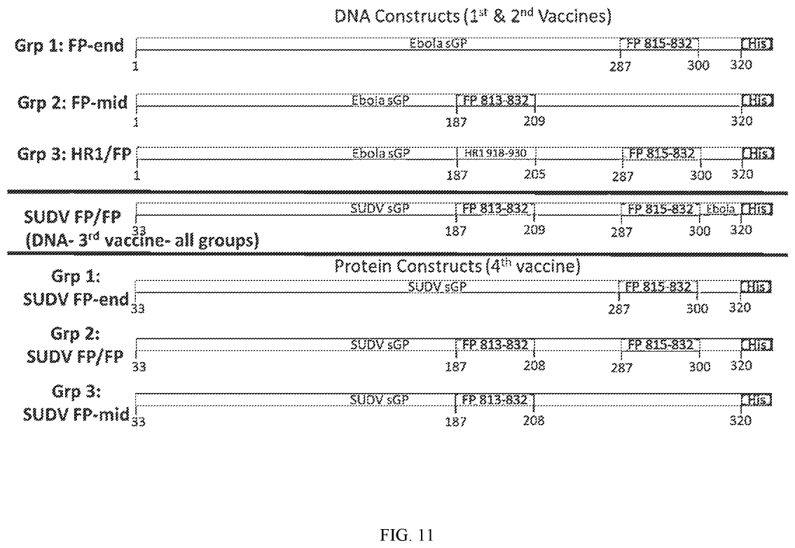

Serum from mice immunized with the following constructs were tested on 50 nanograms/well of pure MERS or COVID spike protein. FP-mid is the construct above with fusion peptide in the middle of sGP. FP-end has the fusion peptide inserted near the end of sGP, with FP amino acids 815-832 replacing sGP residues 288-299. HR1/FP has residues from hairpin 1 (HR1) of COVID spike inserted into the middle of sGP and residues from FP of COVID spike inserted into the end (FP 815-832 replaces sGP 288-299; HR1 918-930 replaces sGP 188-204). A few of the FP-end and HR1/FP samples did react with spike but not as well as FP-mid. This demonstrates that multiple sites on sGP are compatible with insertion of peptides for the generation of vaccines targeted against specific epitopes of COVID spike protein. (FIG. 7)

What is claimed is:

1. A fusion protein comprising a heterologous sequence inserted in the middle of a filovirus secreted glycoprotein (sGP).

2. The fusion protein of claim 1 wherein the filovirus is selected form Ebola virus (EBOV), Sudan virus (SUDV); Bundibugyo virus (BDBV); Tai Forest virus (TAFV); and Lloviu virus (LLOV).

3. The fusion protein of claim 1, wherein the heterologous sequence is inserted between amino acids corresponding to amino acids 188 and 213 of Ebola sGP.

4. The fusion protein of claim 1, wherein the heterologous sequence is inserted after the amino acid corresponding to amino acid 300 in the Ebola virus sGP.

5. The fusion protein of claim 1, wherein the heterologous sequence is a microbial sequence, viral sequence, bacterial sequence, or parasite sequence.

6. The fusion protein of claim 5, wherein the viral sequence is a viral spike protein sequence, a viral heptad repeat (HR) region sequence, a viral HR1 (heptad repeat 1) region sequence, a viral HR2 (heptad repeat 2) region sequence, a viral membrane-proximal extracellular region (MPER) sequence, or a viral surface glycoprotein sequence.

7. The fusion protein of claim 5, wherein the viral sequence is a coronavirus sequence, an influenza virus sequence, an influenza virus hemagglutinin (HA) or neuraminidase (NA) sequence, a Lassa Fever virus sequence, a Lassa Fever virus F protein sequence, a human immunodeficiency virus sequence, a human immunodeficiency virus glycoprotein gp160 sequence, a respiratory syncytial virus sequence, a respiratory syncytial virus surface glycoproteins F or HN sequence, a Nipah virus sequence, a Nipah virus surface glycoprotein G or F sequence, a Hendra virus sequence, a Hendra virus surface glycoproteins G and F, or fragments thereof.

8. A fusion protein of claim 1 wherein the heterologous sequence is a coronavirus sequence comprises an amino acid sequence selected from RSFIEDLLFNKVTLADAGF (SEQ ID NO: 21), ENQKLIANQFNSAI (SEQ ID NO: 11), and LNESLIDLQELGKYE (SEQ ID NO: 22).

9. The fusion protein of claim 1, which has greater than 70% identity to SEQ ID NO: 5, SEQ ID NO: 1, SEQ ID NO: 9, SEQ ID NO: 12, SEQ ID NO: 17, SEQ ID NO: 19, or SEQ ID NO: 20.

10. The fusion protein of claim 5, comprising SEQ ID NO: 5, SEQ ID NO: 1, SEQ ID NO: 9, SEQ ID NO: 12, SEQ ID NO: 17, SEQ ID NO: 19, or SEQ ID NO: 20.

11. A nucleic acid encoding a fusion protein in operable combination with a promoter, wherein the fusion protein comprise a heterologous sequence inserted in the middle of Filovirus secreted glycoprotein (sGP) of claim 1.

12. The nucleic acid of claim 11 which is DNA or RNA.

13. A vector comprising a nucleic acid as in claim 11.

14. An expression system comprising a vector or nucleic acid encoding a fusion protein of claim 1.

15. A method of preventing an infection or reducing the symptoms of an infection comprising administering to a subject in need thereof an effective amount of a fusion protein of claim 1 optionally in combination with an adjuvant.

16. A method of preventing an infection or reducing the symptoms of an infection or comprising administering to a subject in need thereof an effective amount of a vector or nucleic acid encoding a fusion protein in operable combination with a promoter, wherein the fusion protein is as provided for in claim 1 optionally in combination with an adjuvant.


SEQUENCE LISTING (30 sequences)

SEQ ID NO: 1 - protein, 326 aa, Artificial
  Synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGERSFIEDLLFNKVTLADAGKFAVKSCLSQLYQTEPKTSVV

SEQ ID NO: 2 - protein, 6 aa, Artificial
  Synthetic construct
DTTIGE

SEQ ID NO: 3 - protein, 6 aa, Artificial
  Synthetic construct
KFAVKS

SEQ ID NO: 4 - protein, 18 aa, Artificial
  Synthetic construct
RSFIEDLLFNKVTLADAG

SEQ ID NO: 5 - protein, 319 aa, Artificial
  Synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPSKRSFIEDLLFNKVTLADAGPSSGYYSTTIRYQATGFGTNETEYLFEVDNLTYVQLESRFTPQFLLQLNETIYTSGKRSNTTGKLIWKVNPEIDTTIGEWAFWETKKTSLEKFAVKSCLSQLYQTEPKTSVV

SEQ ID NO: 6 - protein, 6 aa, Artificial
  Synthetic construct
AFLILP

SEQ ID NO: 7 - protein, 6 aa, Artificial
  Synthetic construct
PSSGYY

SEQ ID NO: 8 - protein, 20 aa, Artificial
  Synthetic construct
SKRSFIEDLLFNKVTLADAG

SEQ ID NO: 9 - protein, 323 aa, Artificial
  Synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPENQKLIANQFNSAIATEDPSSGYYSTTIRYQATGFGTNETEYLFEVDNLTYVQLESRFTPQFLLQLNETIYTSGKRSNTTGKLIWKVNPEIDTTIGERSFIEDLLFNKVTLADAGKFAVKSCLSQLYQTEPKTSVV

SEQ ID NO: 10 - protein, 6 aa, Artificial
  Synthetic construct
ATEDPS

SEQ ID NO: 11 - protein, 14 aa, Artificial
  Synthetic construct
ENQKLIANQFNSAI

SEQ ID NO: 12 - protein, 315 aa, Artificial
  Synthetic construct
MKFLVNVALVFMVVYISYIYAAMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILASKRSFIEDLLFNKVTLADAGTSSYYATSYLEYEIENFGAQHSTTLFKIDNNTFVRLDRPHTPQFLFQLNDTIHLHQQLSNTTGRLIWTLDANINADIGERSFIEDLLFNKVTLADAGKFAVKSCLSQLYQTEPKTSVV

SEQ ID NO: 13 - protein, 11 aa, Artificial
  Synthetic construct
MKFLVNVALVF

SEQ ID NO: 14 - protein, 6 aa, Artificial
  Synthetic construct
TSSYYA

SEQ ID NO: 15 - protein, 6 aa, Artificial
  Synthetic construct
NADIGE

SEQ ID NO: 16 - protein, 21 aa, Artificial
  Synthetic construct
KFAVKSCLSQLYQTEPKTSVV

SEQ ID NO: 17 - protein, 317 aa, Artificial
  Synthetic construct
MKFLVNVALVFMVVYISYIYAAMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGERSFIEDLLFNKVTLADAGNYVEKSCLSKLYRSTRQKTMMR

SEQ ID NO: 18 - protein, 6 aa, Artificial
  Synthetic construct
NYVEKS

SEQ ID NO: 19 - protein, 316 aa, Artificial
  Synthetic construct
MKFLVNVALVFMVVYISYIYAAMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILASKRSFIEDLLFNKVTLADAGTSSYYATSYLEYEIENFGAQHSTTLFKIDNNTFVRLDRPHTPQFLFQLNDTIHLHQQLSNTTGRLIWTLDANINADIGERSFIEDLLFNKVTLADAGNYVEKSCLSKLYRSTRQKTMMR

SEQ ID NO: 20 - protein, 310 aa, Artificial
  Synthetic construct
MKFLVNVALVFMVVYISYIYAAMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILASKRSFIEDLLFNKVTLADAGTSSYYATSYLEYEIENFGAQHSTTLFKIDNNTFVRLDRPHTPQFLFQLNDTIHLHQQLSNTTGRLIWTLDANINADIGEWAFWENKKISPNNYVEKSCLSKLYRSTRQKTMMR

SEQ ID NO: 21 - protein, 19 aa, Artificial
  Synthetic construct
RSFIEDLLFNKVTLADAGF

SEQ ID NO: 22 - protein, 15 aa, Artificial
  Synthetic construct
LNESLIDLQELGKYE

SEQ ID NO: 23 - protein, 332 aa, Artificial
  Synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGERSFIEDLLFNKVTLADAGKFAVKSCLSQLYQTEPKTSVVHHHHHH

SEQ ID NO: 24 - protein, 325 aa, Artificial
  Synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPSKRSFIEDLLFNKVTLADAGPSSGYYSTTIRYQATGFGTNETEYLFEVDNLTYVQLESRFTPQFLLQLNETIYTSGKRSNTTGKLIWKVNPEIDTTIGEWAFWETKKTSLEKFAVKSCLSQLYQTEPKTSVVHHHHHH

SEQ ID NO: 25 - protein, 329 aa, Artificial
  Synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPENQKLIANQFNSAIATEDPSSGYYSTTIRYQATGFGTNETEYLFEVDNLTYVQLESRFTPQFLLQLNETIYTSGKRSNTTGKLIWKVNPEIDTTIGERSFIEDLLFNKVTLADAGKFAVKSCLSQLYQTEPKTSVVHHHHHH

SEQ ID NO: 26 - protein, 321 aa, Artificial
  Synthetic construct
MKFLVNVALVFMVVYISYIYAAMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILASKRSFIEDLLFNKVTLADAGTSSYYATSYLEYEIENFGAQHSTTLFKIDNNTFVRLDRPHTPQFLFQLNDTIHLHQQLSNTTGRLIWTLDANINADIGERSFIEDLLFNKVTLADAGKFAVKSCLSQLYQTEPKTSVVHHHHHH

SEQ ID NO: 27 - protein, 323 aa, Artificial
  Synthetic construct
MKFLVNVALVFMVVYISYIYAAMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGERSFIEDLLFNKVTLADAGNYVEKSCLSKLYRSTRQKTMMRHHHHHH

SEQ ID NO: 28 - protein, 322 aa, Artificial
  Synthetic construct
MKFLVNVALVFMVVYISYIYAAMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILASKRSFIEDLLFNKVTLADAGTSSYYATSYLEYEIENFGAQHSTTLFKIDNNTFVRLDRPHTPQFLFQLNDTIHLHQQLSNTTGRLIWTLDANINADIGERSFIEDLLFNKVTLADAGNYVEKSCLSKLYRSTRQKTMMRHHHHHH

SEQ ID NO: 29 - protein, 316 aa, Artificial
  Synthetic construct
MKFLVNVALVFMVVYISYIYAAMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILASKRSFIEDLLFNKVTLADAGTSSYYATSYLEYEIENFGAQHSTTLFKIDNNTFVRLDRPHTPQFLFQLNDTIHLHQQLSNTTGRLIWTLDANINADIGEWAFWENKKISPNNYVEKSCLSKLYRSTRQKTMMRHHHHHH

SEQ ID NO: 30 - protein, 6 aa, Artificial
  Synthetic construct
AFLILA